(19) United States
(12) Patent Application Publication
(10) Pub. No.: US 2004/0241670 A1
(43) Pub. Date: Dec. 2, 2004

(54) Method and system for partitioning pixels in a scanned image of a microarray into a set of feature pixels and a set of background pixels

(76) Inventors: Srinka Ghosh, San Francisco, CA (US); Michael P. Caren, Palo Alto, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/453,254

(22) Filed: Jun. 2, 2003

(51) Int. Cl. C12Q 1/68; G06F 19/00; G01N 33/48; G01N 33/50

(52) U.S. Cl. 435/6; 702/20

(57) ABSTRACT

Method and system for partitioning pixels of a region within a scanned, digital image of a microarray into a set of feature pixels and a set of background pixels based on a difference between the intensity variance or noise within a subregion of pixels corresponding to a feature and the variance of background pixels. In one embodiment of the present invention, a standard deviation and mean for the pixel intensities within a microarray-image region are computed, assuming a normal distribution for the noise, and are used to compute a one-dimensional computed probability distribution function for the pixels in the microarray-image region. The one-dimensional, computed probability distribution is generally bimodal, with pixels within a first peak at a lower-probability region of the computed probability distribution corresponding to feature pixels and pixels within a second, larger peak at a relatively larger computed probability corresponding to background pixels. A threshold between the first peak and second peak is used to threshold, or partition, the pixels of the microarray-image region into a set of feature pixels and a set of background pixels.

### TECHNICAL FIELD

The present invention is related to processing of microarray data and, in particular, to a method and system for partitioning pixels in a digital image of a microarray into a set of feature pixels and a set of background pixels.

### BACKGROUND OF THE INVENTION

The present invention is related to methods and systems for determining which pixels, in a digital image of a microarray, are associated with features of the microarray, and which pixels are background pixels associated with inter-feature regions of a microarray. A general background of microarray technology is first provided, in this section, to facilitate discussion of microarray-data processing, in following subsections. It should be noted that microarrays are also referred to as “microarrays” and simply as “arrays.” These alternate terms may be used interchangeably in the context of microarrays and microarray technologies. Art described in this section is not admitted to be prior art to this application.

Array technologies have gained prominence in biological research and are likely to become important and widely used diagnostic tools in the healthcare industry. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (I) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thymidilate subunits in one strand are generally paired with deoxy-adenylate subunits from the other strand. However, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

One, two, or more than two data subsets within a data set can be obtained from a single microarray by scanning the microarray for one, two or more than two types of signals. Two or more data subsets can also be obtained by combining data from two different arrays. When optical scanning is used to detect fluorescent or chemiluminescent emission from chromophore labels, a first set of signals, or data subset, may be generated by scanning the microarray at a first optical wavelength, a second set of signals, or data subset, may be generated by scanning the microarray at a second optical wavelength, and additional sets of signals may be generated by scanning the molecular at additional optical wavelengths. Different signals may be obtained from a microarray by radiometric scanning to detect radioactive emissions one, two, or more than two different energy levels. Target molecules may be labeled with either a first chromophore that emits light at a first wavelength, or a second chromophore that emits light at a second wavelength. Following hybridization, the microarray can be scanned at the first wavelength to detect target molecules, labeled with the first chromophore, hybridized to features of the microarray, and can then be scanned at the second wavelength to detect target molecules, labeled with the second chromophore, hybridized to the features of the microarray. In one common microarray system, the first chromophore emits light at a red visible-light wavelength, and the second chromophore emits light at a green, visible-light wavelength. The data set obtained from scanning the microarray at the red wavelength is referred to as the “red signal,” and the data set obtained from scanning the microarray at the green wavelength is referred to as the “green signal.” While it is common to use one or two different chromophores, it is possible to use one, three, four, or more than four different chromophores and to scan a microarray at one, three, four, or more than four wavelengths to produce one, three, four, or more than four data sets.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical, alphanumerical pixel intensities, or any of many other computer-readable data sets. An initial series of steps employed in processing scanned, digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

The techniques described above generally rely on discriminating feature pixels from background pixels on the basis of differences in intensity between feature pixels and background pixels. The centroids of ROIs are moments of intensity in a two-dimensional pixel space. Unfortunately, there are many cases where discriminating features based on intensity leads to inaccurate and even grossly inaccurate partitioning of pixels between feature pixels and background pixels, and may lead to an inability to identify feature pixels, especially in the case of low-signal features surrounded by noisy background pixels.

FIGS. 8A-E illustrate feature-pixel/background-pixel partitioning problems when the partitioning is based on feature-pixel/background-pixel intensity differentials. FIG. 8A shows a 25×25 pixel region of a digital image of a microarray. Each two-digit number in the two-dimensional array of numbers in FIG. 8A, such as two-digit number “02” 802, represents the intensity associated with a pixel. The intensities are displayed in a two-dimensional array coincident with the two-dimensional array of pixels in the microarray-image region and referenced by x and y coordinates based on an x axis 804 and a y axis 806. Thus, the intensity of the pixel at location (0,0) with respect to the x and y axes is shown as the two-digit number “02” 802 in FIG. 8A. The intensity of pixel (24, 24) 808 is represented by the two-digit number “07.” The two-digit numbers represent the pixel intensity divided by 100. The pixel intensities shown in the region illustrated in FIG. 8A were generated computationally in order to provide a hypothetical example, variants of which are used throughout the subsequent discussion of various embodiments of the present invention. In these subsequent discussions, a number of figures similar to FIG. 8A are referenced, and the illustration conventions described above with respect to FIG. 8A are used, as well, in these subsequently referenced figures.

Visual inspection of the pixel intensities in the microarray-image region shown in FIG. 8A reveals a roughly disk-shaped region with relatively high intensities surrounded by an annulus of intermediate intensities that together represent the image of a feature. FIG. 8B shows the region of a hypothetical microarray illustrated in FIG. 8A overlaid with two roughly circular line boundaries of the disk-shaped high-intensity and intermediate-intensity portions of the microarray-image region. The highest-intensity portion of the microarray-image region 810 shows intensities varying between 4800 and 5099, since integer division by 100 is employed to produce the two-digit representations of intensities, and the intermediate-intensity annular portion 812 or the microarray-image region surrounding the high-intensity portion contains intensities between 2300 and 2799. The remaining portion of the microarray-image region 814 contains relatively low-intensity pixels, having an average intensity of 1000, and corresponds to the background of the microarray-image region. Thus, as shown in FIGS. 8A-B, feature pixels in the microarray-image region illustrated in FIGS. 8A-B can be readily chosen from the microarray-image region by choosing a threshold intensity, and allocating those pixel intensities below the threshold intensity to a background-pixel partition, and allocating those pixels above a threshold pixel intensity to a feature-pixel partition. As described above, currently employed techniques computationally identify an intensity moment, or centroid, in the subregion and then identify a feature region approximately coincident with the centroid based on intensity thresholding.

FIG. 8C shows a second hypothetical region of a microarray. This second hypothetical microarray-image region is identical to the microarray-image region illustrated in FIGS. 8A-B, except that the average intensity of the high-intensity, central feature pixels is only 2000, rather than 5000. Note that, although the feature can still be visually distinguished by carefully examining the two-dimensional array of pixel intensities, the task of visually discriminating feature pixels from background pixels is significantly more difficult. FIG. 8D shows the high-intensity, central feature portion of the microarray-image region, illustrated in FIG. 8C, outlined. FIG. 8E shows a microarray-image region similar to those shown in FIGS. 8A-D, with exception that the average intensities of feature pixels within the high-intensity, central portion of the feature is only 1500. Thus, in FIG. 8E, the high-intensity, central feature pixels have an average intensity only 1.5 times that of the background pixels. Note that it is quite difficult to distinguish feature pixels from background pixels based on intensity alone. Note also that these hypothetical examples are, in many ways, best-case types of examples, in that the feature region is regularly shaped and relatively uniform in intensity. In actual microarray data, features may be asymmetrical, may contain highly non-uniform intensities due to a large number of different possible procedural, experimental, and instrumental errors and instabilities, and may be substantially offset from their expected positions within the general grid-like, regular pattern in which features are deposited. All of these effects can lead to difficulties in employing the currently used, intensity-based feature-pixel/background-pixel partitioning methods described above. For these reasons, there is a need for other methods for partitioning pixels within a digital image of a microarray into sets of feature pixels and background pixels.

### SUMMARY OF THE INVENTION

In one embodiment of the present invention, the pixels of a region within a digital image of a microarray are partitioned into a set of feature pixels and a set of background pixels based on a difference between the intensity variance within a subregion of pixels corresponding to a feature and the variance of background pixels. In general, the variance of background pixels is observed to be substantially greater than that of feature pixels. It is also observed that the noise distribution of pixels can be modeled as a Gaussian, or normal, distribution. In a biological system the noise model may follow a Rayleigh or possibly even a gamma distribution. In one embodiment of the present invention, a standard deviation and mean for the pixel intensities within a microarray-image region are computed, and are used to compute a computed noise probability distribution function for the pixels in the microarray-image region. This computed probability distribution is generally bimodal, with pixels within a first peak at a lower-probability region of the computed probability distribution corresponding to feature pixels and pixels within a second, larger peak at a relatively larger computed probability corresponding to background pixels. A threshold probability value is selected from a domain between the first peak and second peak and used to threshold, or partition, the pixels of the microarray-image region into a set of feature pixels and a set of background pixels. In the described embodiments, feature pixels can be successfully differentiated from background pixels without recourse to methods based on pixel intensity differentials between feature pixels and background pixels, general improving the accuracy of the feature-pixel/background-pixel partition.

The computed probability distribution is generally bimodal, with pixels within a first peak at a lower-probability region of the computed probability distribution corresponding to feature pixels and pixels within a second, larger peak at a relatively larger computed probability corresponding to background pixels. A threshold between the first peak and second peak is used to threshold, or partition, the pixels of the microarray-image region into a set of feature pixels and a set of background pixels. In the described embodiments, feature pixels can be successfully differentiated from background pixels without recourse to methods based on differences between the intensities of feature pixels and background pixels, general improving the accuracy of the feature-pixel/background-pixel partition.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIGS. 8A-E illustrate feature-pixel/background-pixel partitioning problems when the partitioning is based on feature-pixel/background-pixel intensity differentials.

FIG. 9 shows a region of a microarray image containing a single feature.

FIG. 10 shows a histogram of the pixel intensities from the microarray-image region shown in FIG. 9.

FIG. 11 shows a one-dimensional histogram of computed probabilities for pixels in the microarray-image region shown in FIG. 9.

FIG. 12 illustrates a two-dimensional, computed-probability space corresponding to the two-dimensional, pixel-intensity space illustrated in FIG. 9.

FIGS. 13A-B show representations of the two-dimensional intensity space and a representation of the two-dimensional computed-probability space, both obtained by thresholding.

FIGS. 14A-B provide flow-control-diagram representations of a feature partitioning method that represents one embodiment of the present invention.

FIGS. 15A-D show the two-dimensional pixel intensity space, the two-dimensional computed probability space corresponding to the pixel intensity space, a thresholded two-dimensional pixel intensity space, and a thresholded two-dimensional computed probability space corresponding to the two-dimensional pixel intensity space, for a feature having a central-region average intensity of 3000, an intermediate-intensity annulus having an average pixel intensity of 1500, and with all other parameters equal to those used to compute the hypothetical microarray-image region shown in FIG. 9.

FIGS. 16A-D provide two-dimensional pixel intensity spaces and computer-probability spaces analogous to those shown in FIGS. 15A-D, with the exception that the average intensity of the central feature pixels is 2000, and that of the intermediate-intensity feature pixels of 1000, with all other computational parameters identical to those used for generating the microarray-image region shown in FIG. 9.

FIGS. 17A-F illustrate the variance-based pixel partitioning method that represents one embodiment of the present invention applied to a hypothetical microarray-image region having an average central-feature intensity of 1500 and an average intermediate-level feature intensity of 750 in a manner analogous to FIGS. 9-13B.

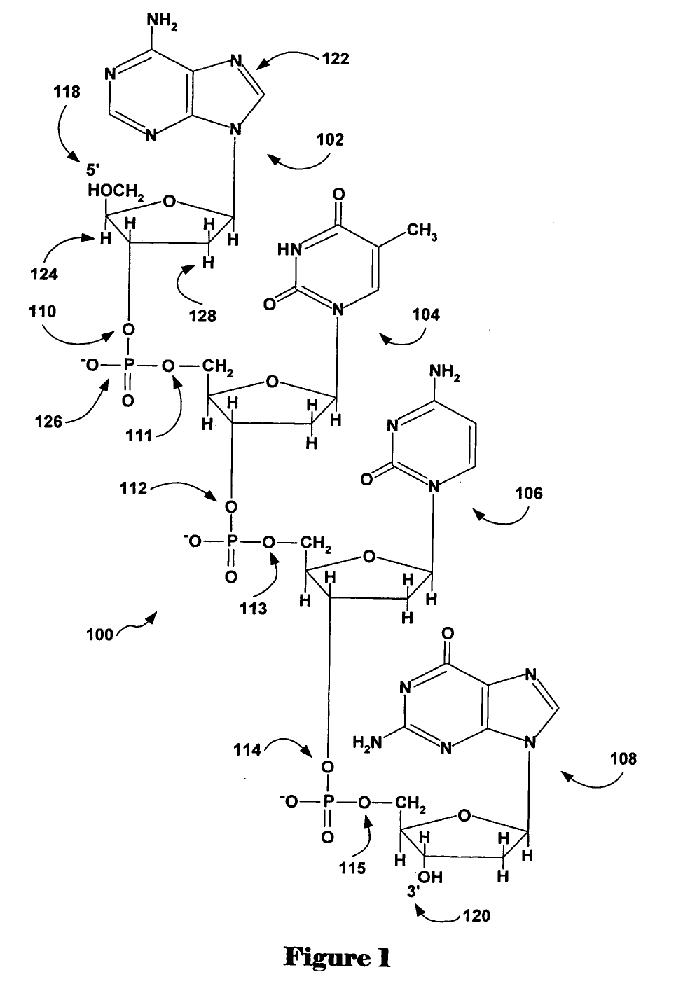

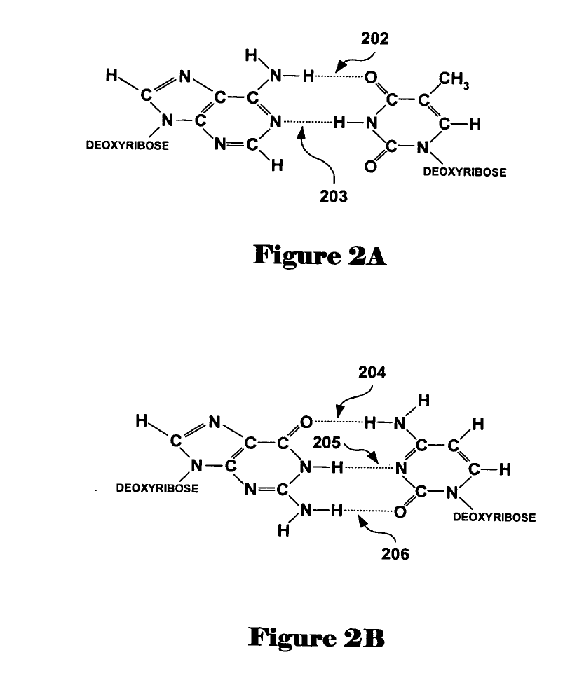

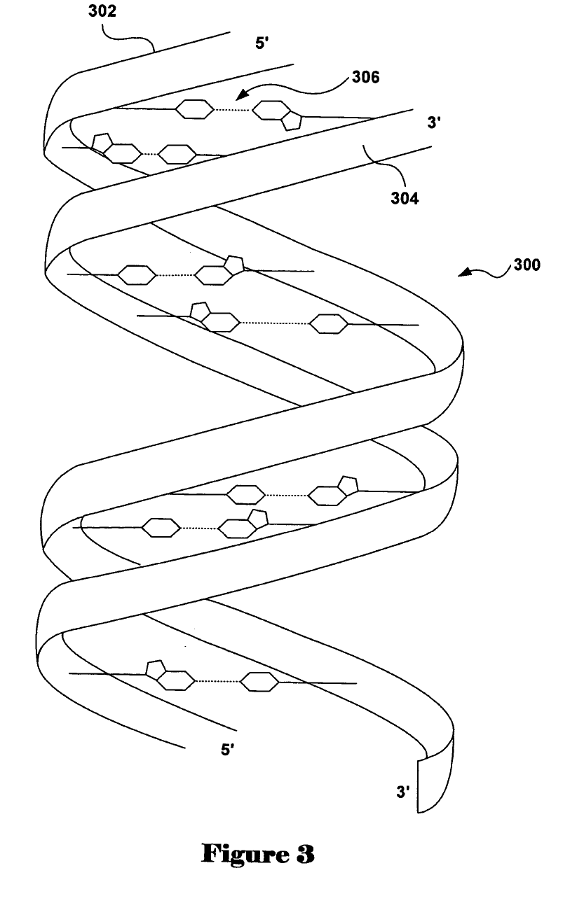

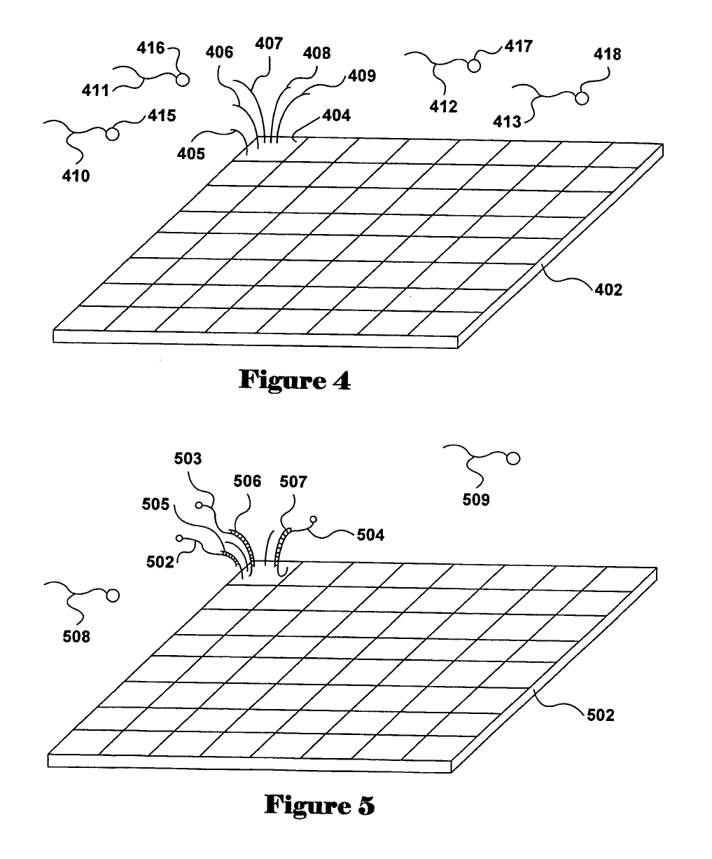

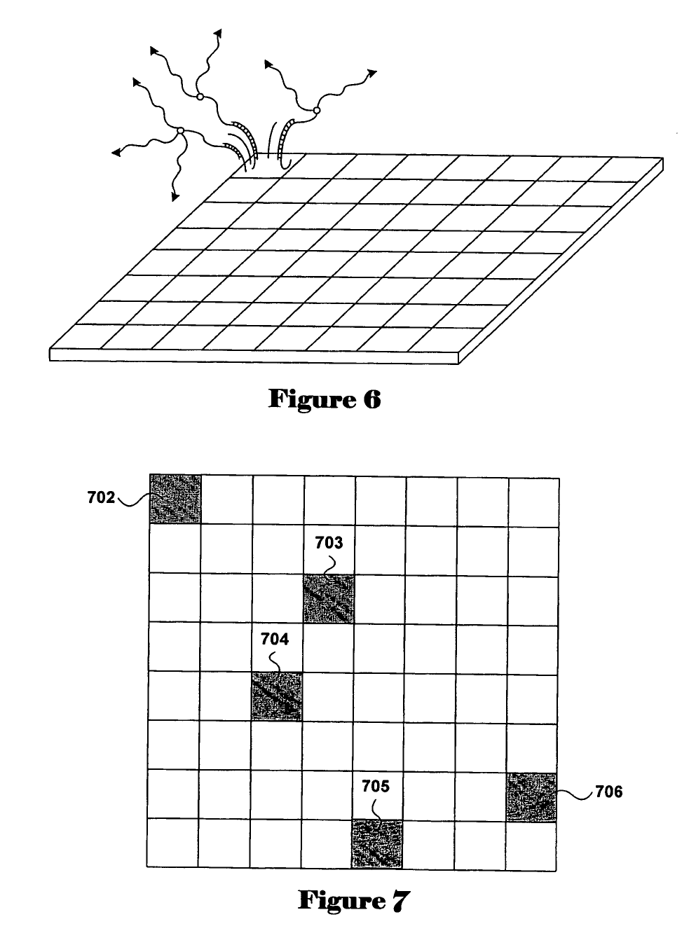

### DETAILED DESCRIPTION OF THE INVENTION

One embodiment of the present invention provides a method and system for discriminating between pixels, in a digital image of a microarray, associated with features and pixels in inter-feature regions of the microarray image referred to as background pixels. In a first subsection, below, additional information about microarrays is provided. Those readers familiar with microarrays may skip over this first subsection. In a second subsection, embodiments of the present invention are provided through examples, graphical representations, and with reference to several flow-control diagrams.

### Additional Information About Molecular Arrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

A microarray is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087,447 “Reading Dry Chemical Arrays Through The Substrate” by Corson et al., and in U.S. Pat. Nos. 6,518,556; 6,486,457; 6,406,849; 6,371,370; 6,355,921; 6,320,196; 6,251,685; and 6,222,664. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

### Embodiments of the Present Invention

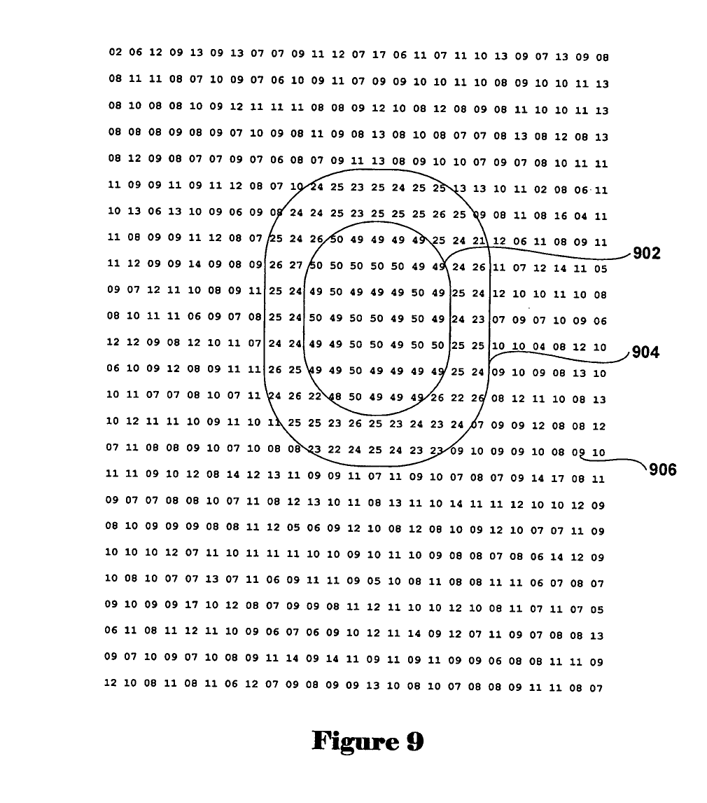

FIG. 9 shows a region of a microarray containing a single feature. The feature includes a central, high-intensity region 902, with an average pixel intensity of 5000, surrounded by an annular, lower-intensity region 904 with an average pixel intensity of 2500. The remaining pixels in the region 906 are background pixels with an average intensity of 1000. Note that the pixel intensities for the hypothetical microarray-image region shown in FIG. 9 were computationally generated to provide examples particularly suited for the following discussion.

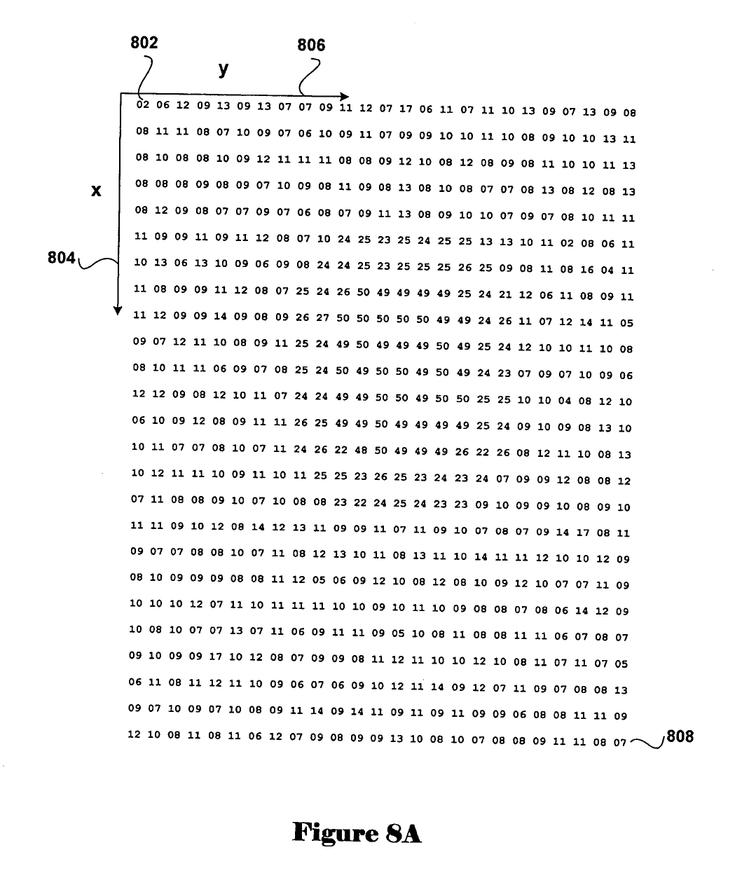

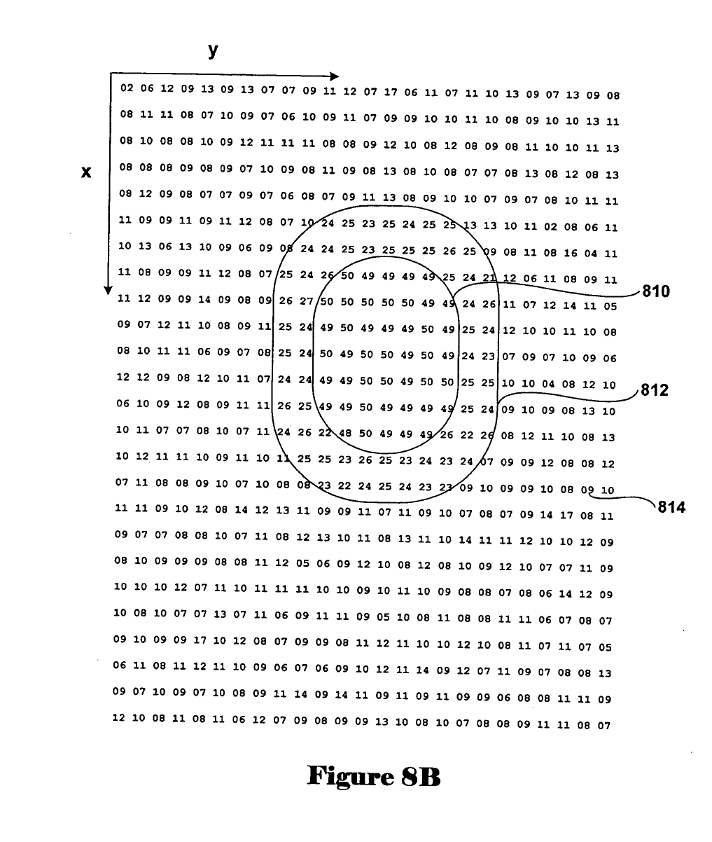

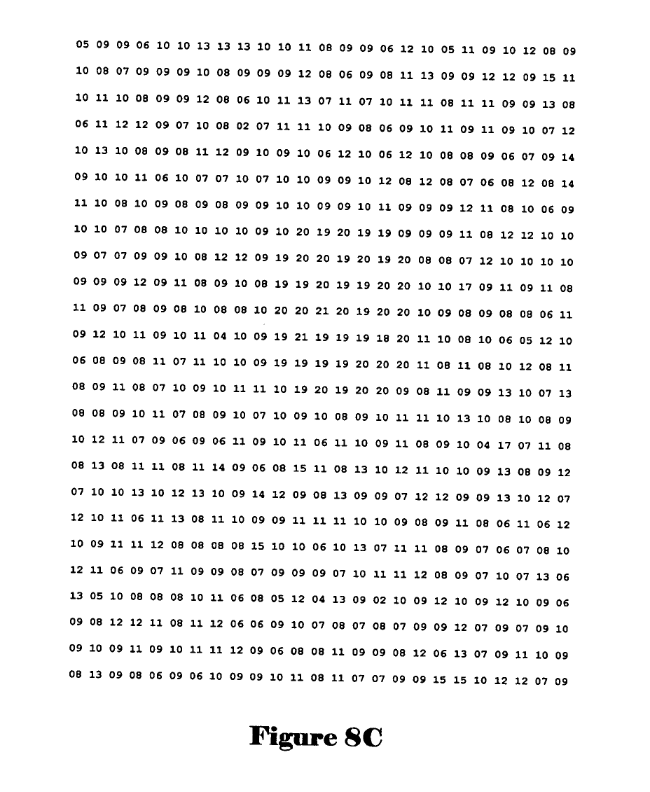

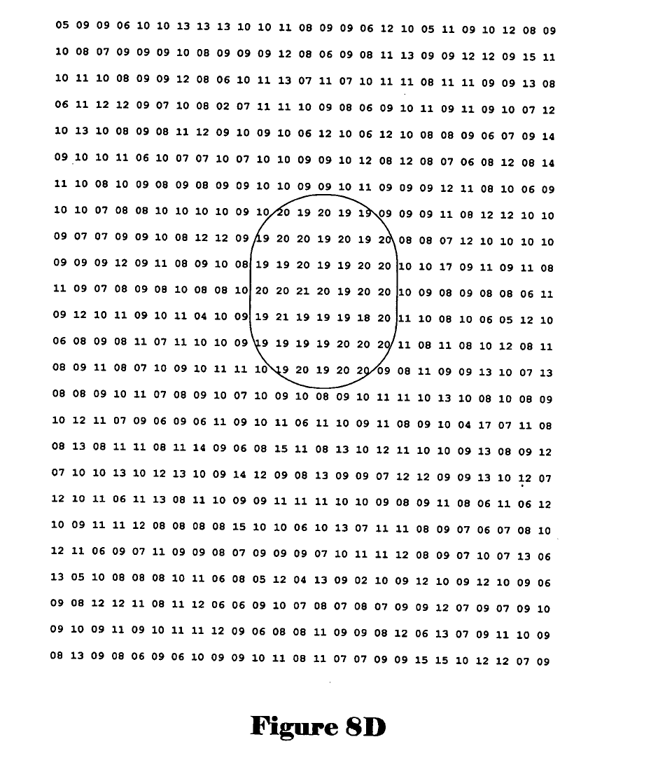

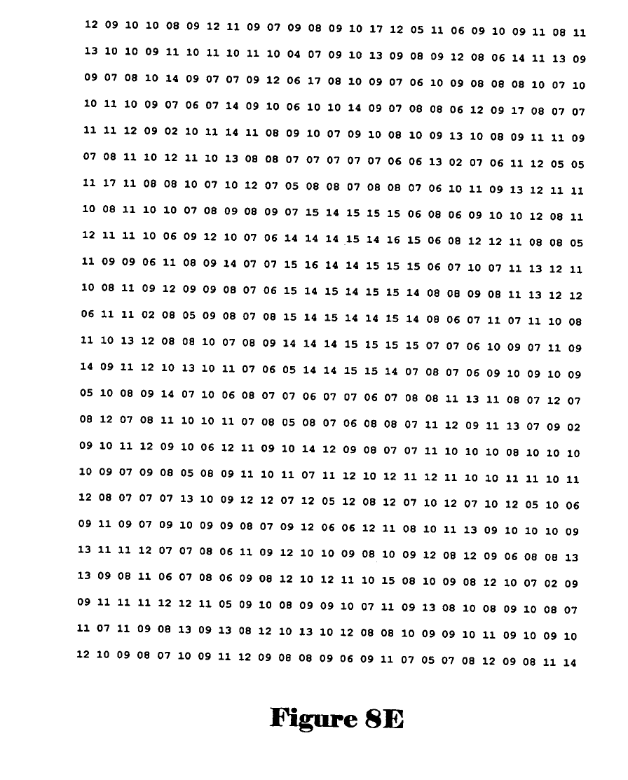

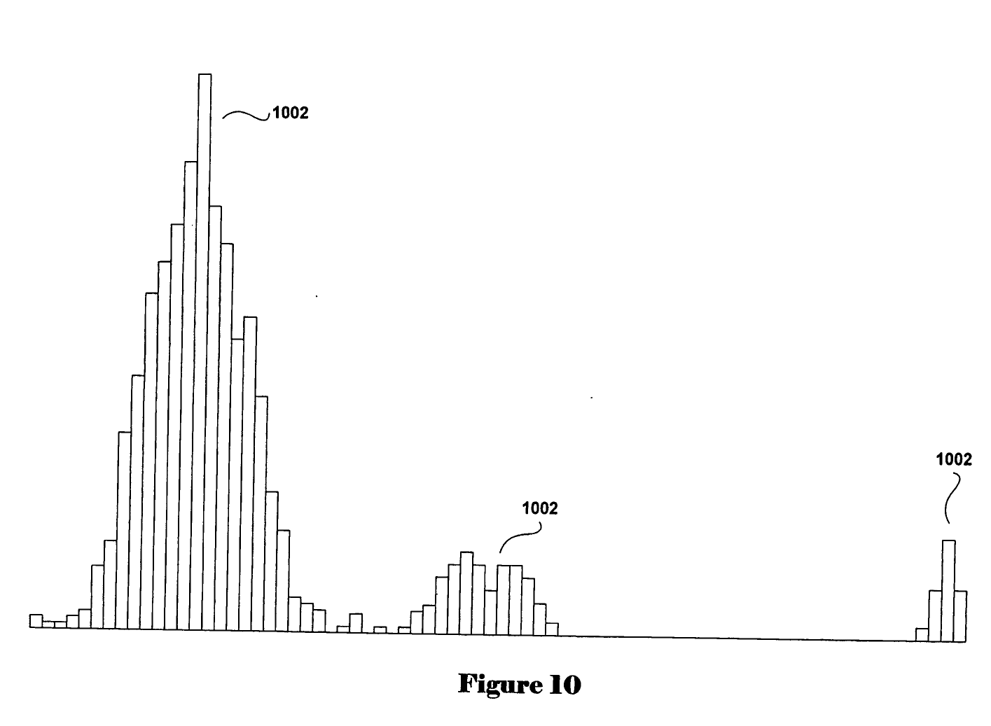

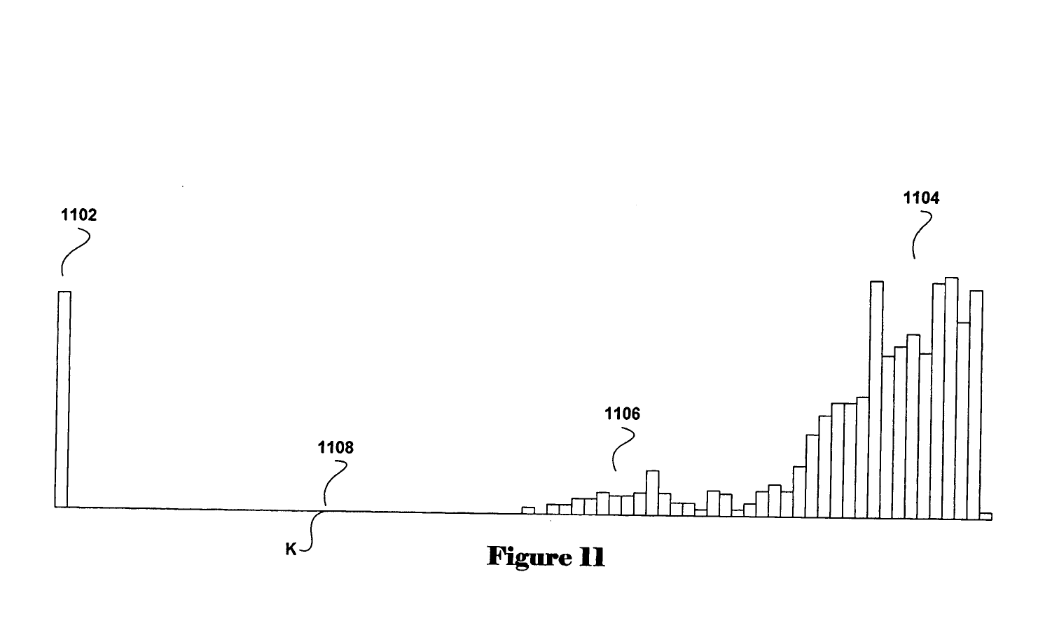

FIG. 10 shows a histogram of the pixel intensities from the microarray-image region shown in FIG. 9. The pixel intensities are cleanly distributed into three peaks: (1) a first, comparatively tall and broad peak 1002, corresponding to background pixels; (2) a second, smaller peak 1004, representing the lower-intensity feature pixels from the lower-intensity annular region (904 in FIG. 9); and (3) a small, high-intensity peak 1006 corresponding to the high-intensity feature pixels within the central portion of the feature (902 in FIG. 9). Thus, as shown in FIG. 10, the feature pixels can be cleanly separated from background pixels based on intensity alone, as demonstrated by the visual separation illustrated in FIGS. 8A-B.

As discussed above, however, partitioning of pixels into a feature-pixel set and a background-pixel set based on intensity differentials can be problematic when real microarray data is processed. Thus, efforts were undertaken to identify a different criterion or criteria by which feature pixels and background pixels can be distinguished. By careful observation and experimentation, the variance of pixel intensities was identified as being a more reliable partitioning criterion.

In order to partition pixels or, equivalently, pixel intensities, based on intensity variance, the mean intensity μ and standard deviation σ, or variance σ² for all of the pixels in a microarray-image region are first computed according to the formulas provided below:

$$\begin{matrix}μ = \frac{1}{{N}_{i} {N}_{j}} {\sum }_{i = 0}^{{N}_{i}} {\sum }_{j = 0}^{{N}_{j}} {I}_{i , j} \\ {σ}^{2} = \frac{1}{{N}_{i} {N}_{j}} {\sum }_{i = 0}^{{N}_{i}} {\sum }_{j = 0}^{{N}_{j}} {( {I}_{i , j} - μ )}^{2} \\ σ = \sqrt{{σ}^{2}}\end{matrix}$$

where I_{i,j} is the intensity associated with the pixel at location (i,j),

Nᵢ is the length, in pixels, of the microarray-image region, and

Nⱼ is the width, in pixels, of the microarray-image region.

Next, assuming that the noise distribution of pixels is roughly Gaussian, or normal, a probability of each pixel intensity can be computed for each pixel in the microarray-image region as follows:

$$p ( {I}_{i , j} ) = \frac{1}{\sqrt{2 π } σ} {ⅇ}^{- ( \frac{{( {I}_{i , j} - μ )}^{2}}{2 {σ}^{2}} )}$$

The computed probabilities of the pixel intensities can be arranged in a one-dimensional histogram, similar to the histogram of intensities shown in FIG. 10. FIG. 11 shows a one-dimensional histogram of computed probabilities for pixels in the microarray-image region shown in FIG. 9. As can be seen in FIG. 11, the computed probability histogram shows two major, well-differentiated peaks. A first, extremely narrow peak 1102 occurs at the low-probability end of the histogram, and a second, relatively broad peak 1104 occurs at the high probability end of the computed-probability histogram. Because the intermediate-intensity annular region of the feature (904 in FIG. 9) was computed to have a somewhat greater variance than the inner, high-intensity region (902 in FIG. 9), a third peak 1106 occurs in the histogram corresponding to the annular-region pixels. In much observed microarray data, computed-probability histograms tend to exhibit two major peaks, one corresponding to feature pixels, and one corresponding to background pixels. However, it may be possible that, as in the hypothetical data shown in FIG. 9, more than two partitions based on more than two distinct variances may be observed.

As shown in FIG. 11, feature pixels can be cleanly differentiated from background pixels based on the departure of the probability of feature-pixel intensities from an assumed normal distribution of pixel intensities within the microarray-image region. In essence, the computed probability histogram represents a transformation from a two-dimensional, pixel-intensity space to a one-dimensional, computed probability space.

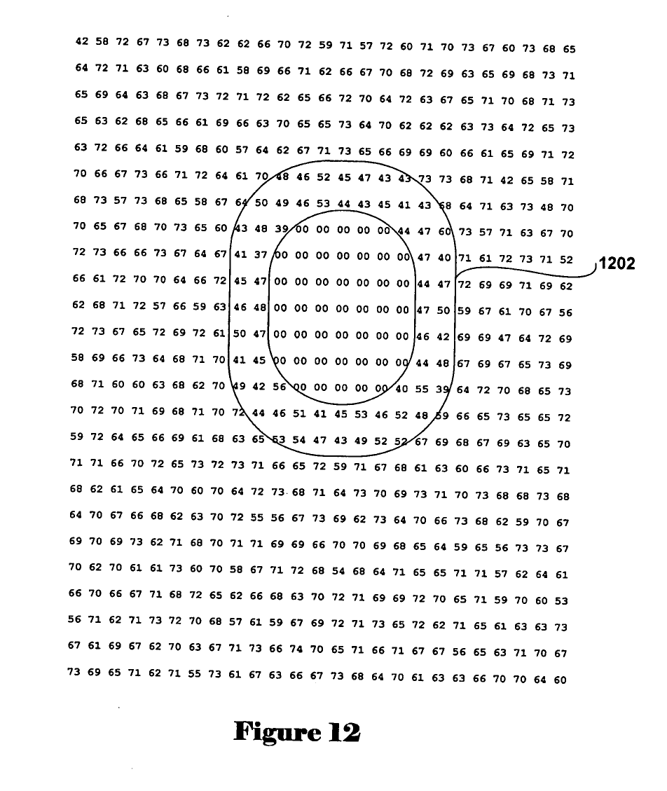

FIG. 12 illustrates a two-dimensional, computed-probability space corresponding to the two-dimensional, pixel-intensity space illustrated in FIG. 9. In FIG. 12, each two-digit number represents the bin number within the one-dimensional, computed probability space (FIG. 11) to which the intensity associated with the corresponding pixel is assigned, based on the computed probability for the intensity. Note that feature 1202 is easily visually distinguished from the background pixels based on the displayed bin numbers, or, equivalently, ordered probability ranges. If a threshold histogram bin k (1108 in FIG. 11) is chosen between the first major peak (1102 in FIG. 11) and the second major peak (1104 in FIG. 11), then the threshold bin can be used to create a binary map from the two-dimensional, computed-probability distribution shown in FIG. 12.

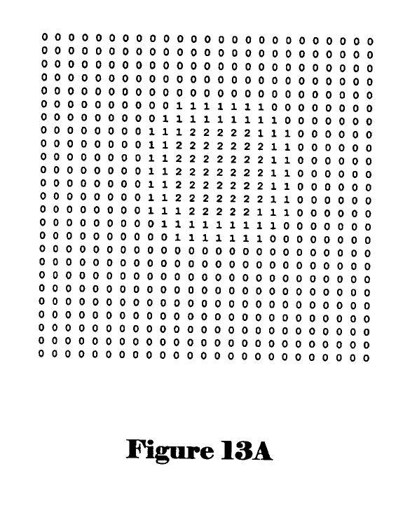

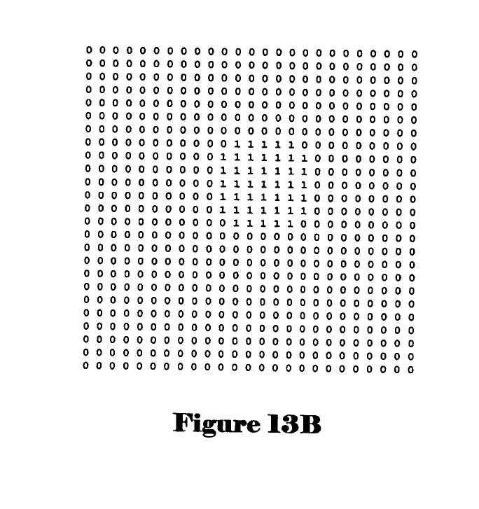

FIGS. 13A-B show representations of the two-dimensional intensity space and a representation of the two-dimensional computed-probability space, both obtained by thresholding. In FIG. 13A, high-intensity pixels are indicated by the digit “2,” intermediate-intensity pixels are indicated by the digit “1,” and low-intensity pixels are indicated by the digit “0.” In FIG. 13B, low probability pixels are indicated by the digit “1,” and comparatively higher-probability pixels are indicated by the digit “0.” As can be seen by comparing FIG. 13A to 13B, the low-probability pixels in FIG. 13B exactly correspond to the high-intensity pixels in FIG. 13A. Thus, by thresholding the two-dimensional computed-probability distribution, shown in FIG. 12, an exact spatial delineation of the central feature portion of the microarray-image region is obtained. Of course, were both the central and annular portions of the feature computed to have similar variances, the annular portion of the feature would also be selected as part of the low-intensity region. Alternatively, several thresholds may be obtained from the computed-probability histogram and used to create a trinary, two-dimensional, thresholded computed-probability space representation, which would then show a lowest-probability region corresponding to the inner portion of the feature, a second lowest-probability region corresponding to the annular portion of the feature, and a comparatively higher-probability region corresponding to background pixels.

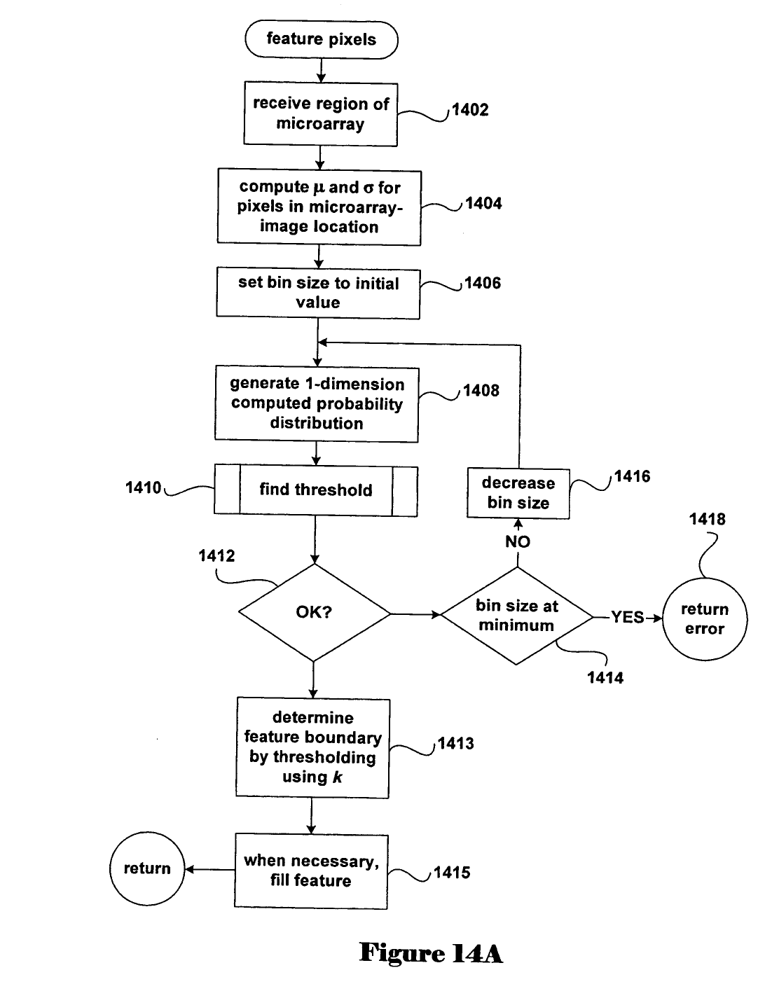

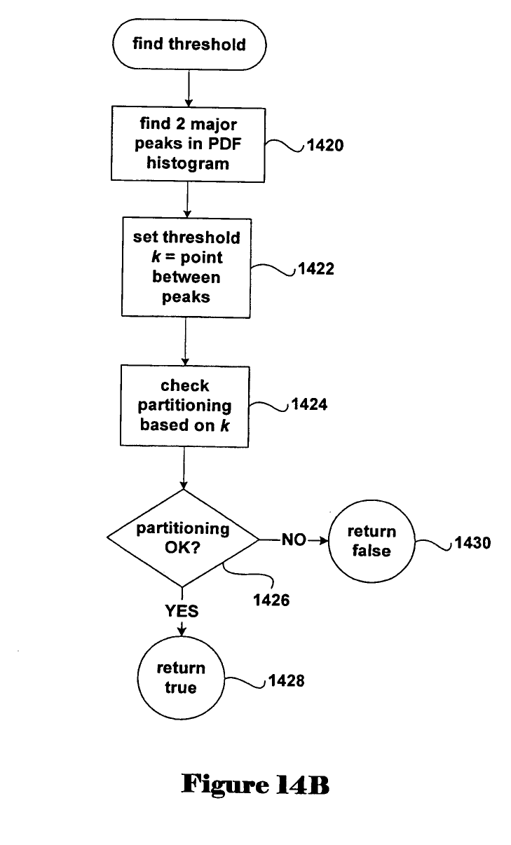

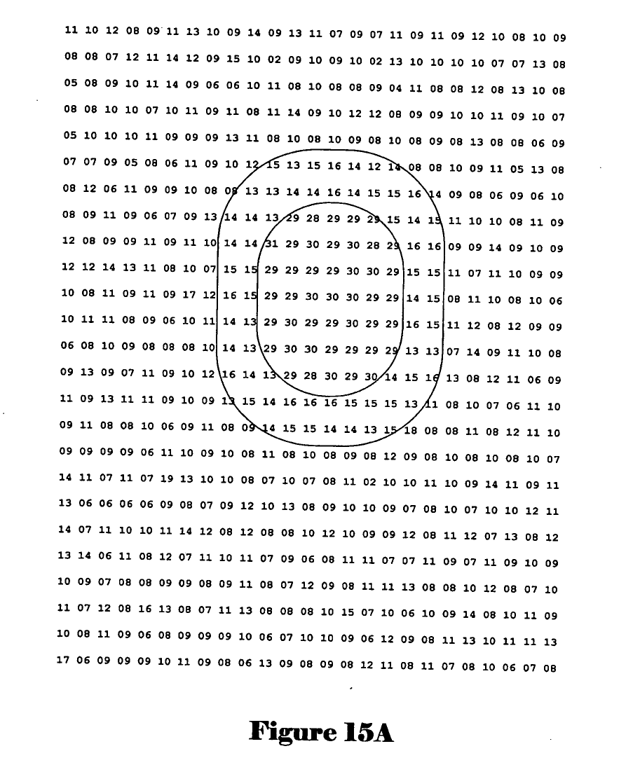

FIGS. 14A-B provide flow-control-diagram representations of a feature partitioning method that represents one embodiment of the present invention. In step 1402 of FIG. 14A, the routine “feature pixels” receives a representation of a microarray-image region. In step 1404, the routine “feature pixels” computes the mean and standard deviation for all of the pixels in the microarray-image region. In step 1406, the routine “feature pixels” sets an initial bin size for generating the computed-probability distribution. In step 1408, the routine “feature pixels” generates a one-dimensional, computed probability space for the pixels, as described above with reference to FIG. 11. In step 1410, the routine “feature pixels” calls the routine “find threshold,” provided in FIG. 14B, to determine a one-dimensional, computed-probability-histogram threshold bin k, to differentiate between feature pixels and background pixels. The routine “find threshold” returns a Boolean value indicating whether or not partitioning based on the threshold k is acceptable. If the partitioning is acceptable, as determined in step 1412, then the routine “feature pixels” determines the feature boundary from a thresholded two-dimensional computed-probability distribution, an example of which is shown in FIG. 13B, in step 1414. Finally, in step 1416, the feature can be filled, when necessary, to create a uniform feature mask that can then be employed in subsequent microarray data processing to distinguish feature pixels from background pixels. If the threshold k does not produce an acceptable partitioning, as determined in step 1412, and if the bin size is not at a minimum bin size, as detected in step 1414, then the routine “feature pixels” decreases the bin size, in step 1416, and control returns to step 1408 for computation of an additional, finer-grained, one-dimensional, computed probability space. If the bin size is already at a minimum bin size, as determined in step 1414, then an error is returned in step 1418.

FIG. 14B is a flow-control diagram for the routine “find threshold.” In step 1420, the routine “find threshold” finds the two major peaks in the one-dimensional computed-probability histogram computed in step 1408 in FIG. 14A. Note, as noted above, if more than two probability peaks are expected, additional peaks may be detected in order to serve as the basis for calculating more than a single threshold. However, in the present description, it is assumed that the probabilities naturally partition into two peaks, as occurs in many microarray data sets, so that only two major peaks need to be found. Peak finding can be accomplished by any of many well-known computational techniques, including computing a local peak metric for each bin of the histogram and choosing the two bins with a highest metric that are spaced a reasonable distance apart. The peak metric may consist of a sum of the differences of the average probability of the bin of interest and those of neighboring bins. Next, in step 1422, the routine “find threshold” sets the threshold k to a point between the two peaks. Again, many different techniques can be used to set the threshold k. It may be set at an arbitrary ratio of the distance between the two peaks times the distance between the two peaks plus the position of the first peak, for example. In other techniques, slopes of the peaks can be calculated to determine their point of intersection with the histogram access, and the threshold k chosen equidistant between the two points of intersection. In some cases, the peaks may overlap, requiring that k be selected as the intersection of the two descending slopes of the peaks. Many other well-known, sophisticated computational techniques for choosing a bin threshold k can be employed. Next, in step 1424, the routine “find threshold” checks that the partitioning based on this threshold k is acceptable. Many different checks are possible. The routine “find threshold,” for example, can check to make sure that k resides at a true minimum between the two greatest peaks in the histogram. Many other qualitative checks for the acceptability of threshold k are possible, and well known in the art. If the partitioning is acceptable, as determined in step 1426, then the routine “find threshold” returns a Boolean value TRUE in step 1428. Otherwise, the routine “find threshold” returns a Boolean value FALSE in step 1430.

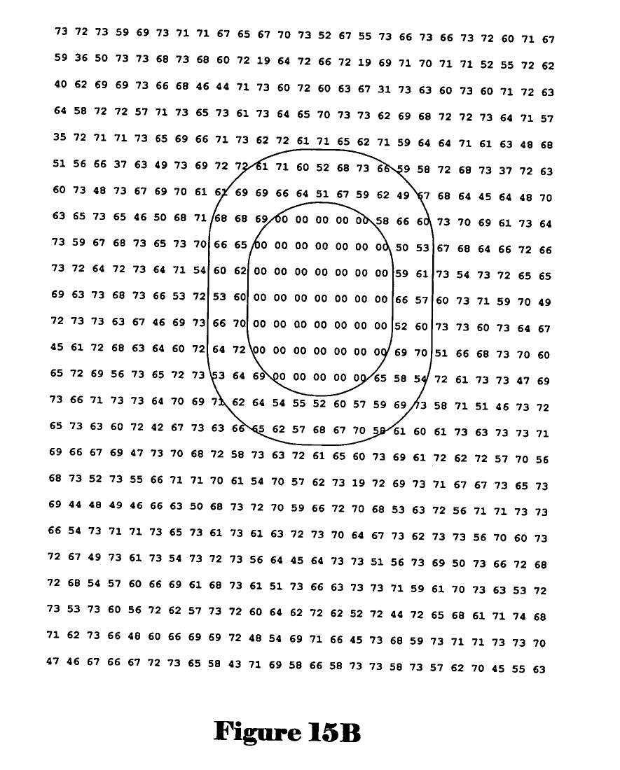

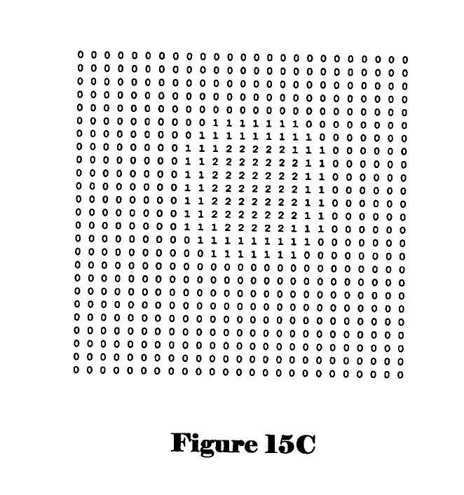

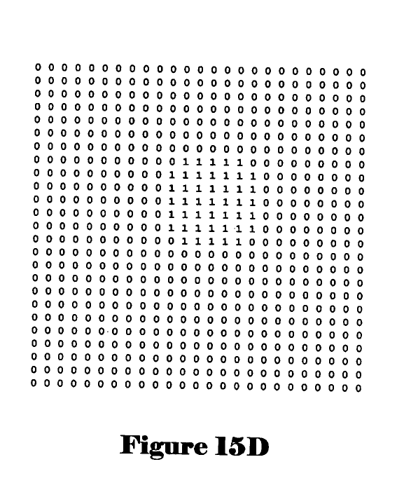

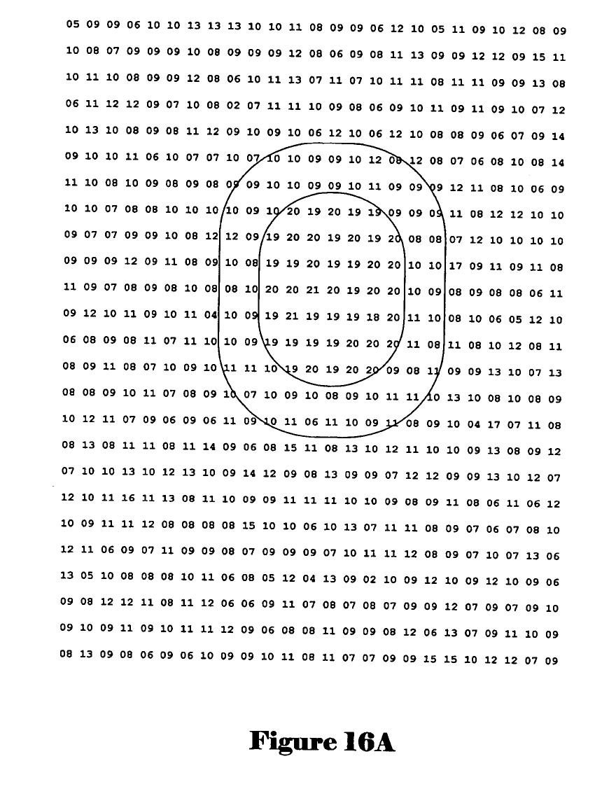

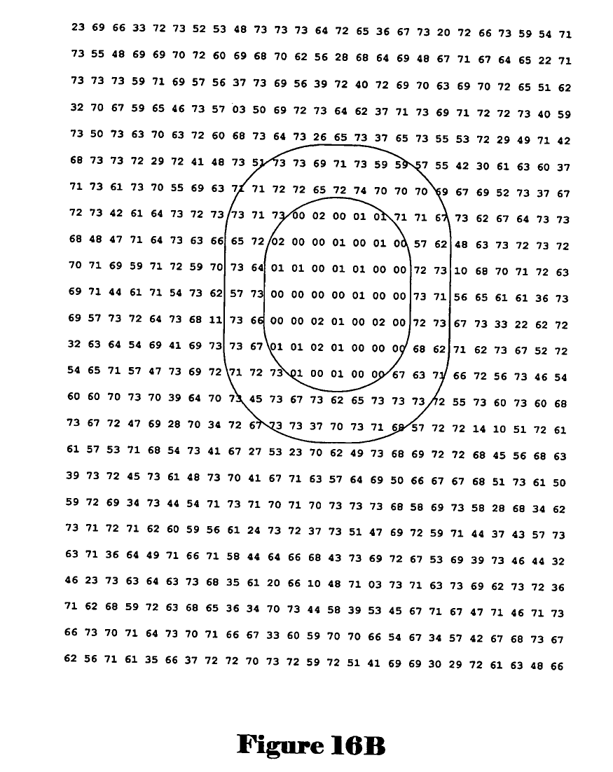

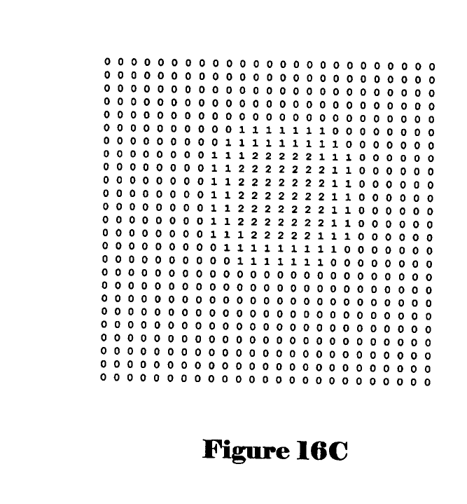

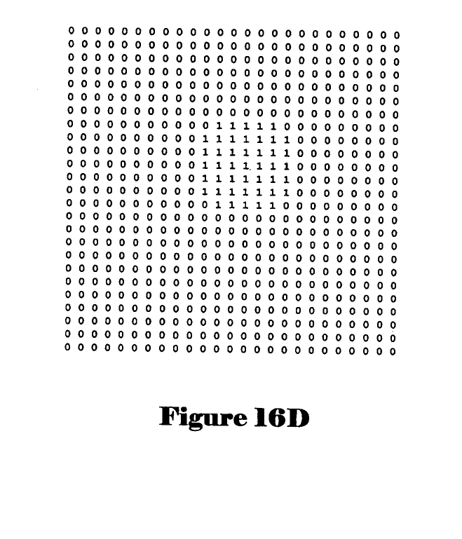

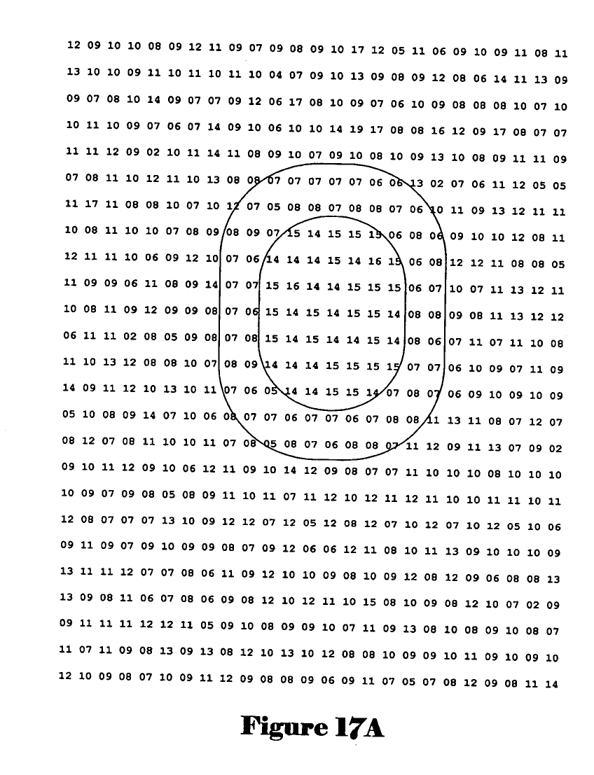

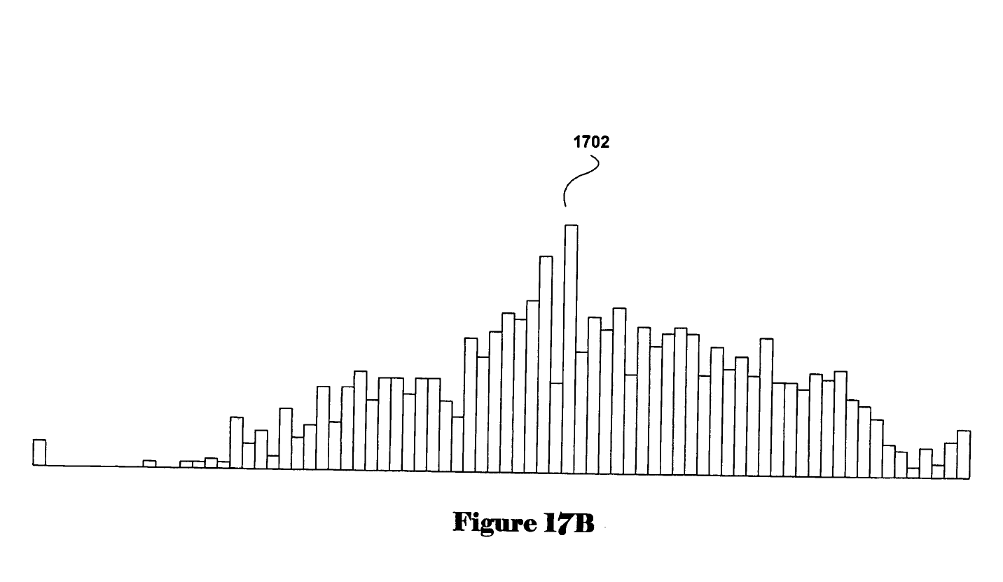

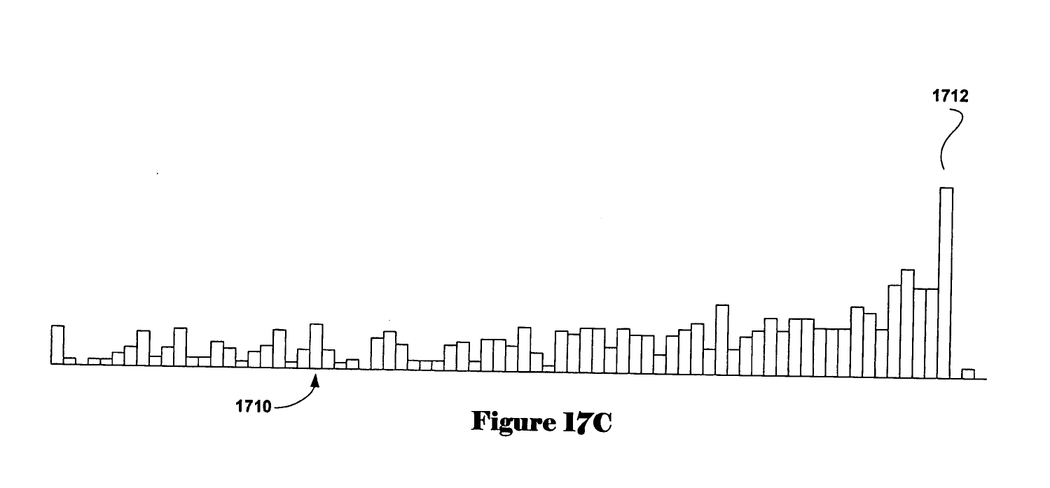

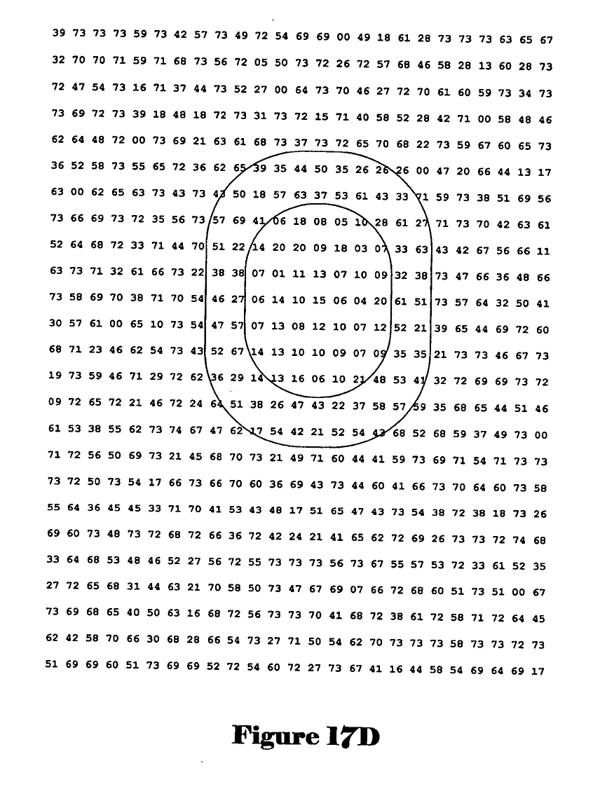

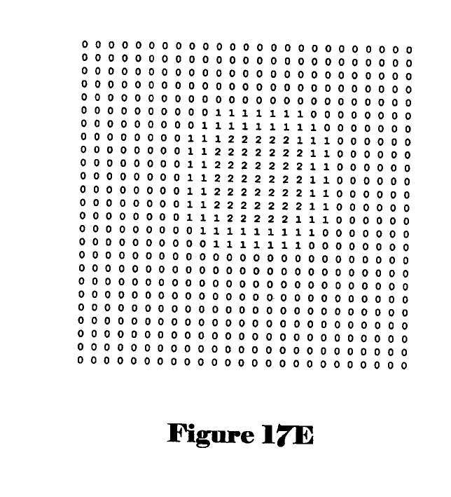

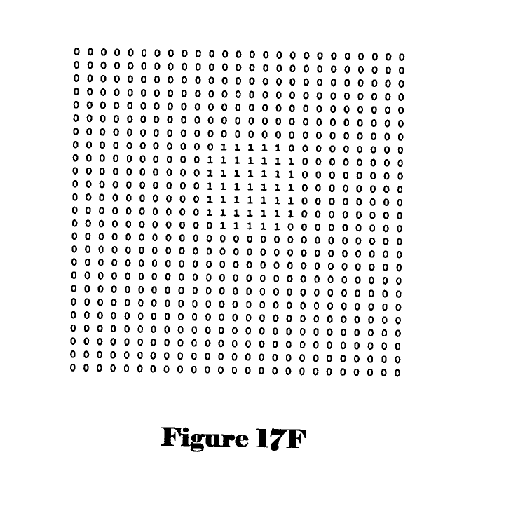

Note that, referring back to FIG. 9, the hypothetical microarray-image region was calculated with a central feature region having an average intensity of 5000 and a standard deviation of 50, the annular, lower-intensity feature region having an average pixel intensity of 2500 and a standard deviation of 100, and the background pixels having an average intensity of 1000, and a standard deviation of 200. FIGS. 15A-D show the two-dimensional pixel intensity space, the two-dimensional computed probability space corresponding to the pixel intensity space, a thresholded two-dimensional pixel intensity space, and a thresholded two-dimensional computed probability space corresponding to the two-dimensional pixel intensity space, for a feature having a central-region average intensity of 3000, an intermediate-intensity annulus having an average pixel intensity of 1500, and with all other parameters equal to those used to compute the hypothetical microarray-image region shown in FIG. 9. As can be seen in the thresholded, two-dimensional computed-probability space, shown in FIG. 15D, the variance-based method that represents one embodiment of the present invention can successfully identity feature pixels when the average feature-pixel intensity exceed that of background pixels by a factor of three. FIGS. 16A-D provide two-dimensional pixel intensity spaces and computer-probability spaces analogous to those shown in FIGS. 15A-D, with the exception that the average intensity of the central feature pixels is 2000 and that of the intermediate-intensity feature pixels of 1000, with all other computational parameters identical to those used for generating the microarray-image region shown in FIG. 9. Again, inspection of the thresholded two-dimensional computer-probability space, shown in FIG. 16D, reveals that even when the average feature intensity exceeds the average background pixel intensity by only a factor of two, the feature pixels are cleanly distinguished by the variance-based method that represents one embodiment of the present invention. FIGS. 17A-F illustrate the variance-based pixel partitioning method that represents one embodiment of the present invention applied to a hypothetical microarray-image region having an average central-feature intensity of 1500 in a manner analogous to FIGS. 9-13B. FIG. 17B shows the intensity distribution for the pixels and the hypothetical microarray-image region shown in FIG. 17A. Note that the pixel intensity distribution is no longer trimodal, or even bimodal. Instead, all of the pixel intensities fall into a very broad, single peak 1702. Thus, as is clear from the pixel-intensity histogram of FIG. 17B, the feature pixels cannot be distinguished from the background pixels by intensity differential, alone. As can be seen in FIG. 17C, the one-dimensional, computed probability space for the microarray-image region shown in FIG. 17A is relatively flat, but two, relatively well-separated peaks 1710 and 1712 can be computationally identified. Thresholding based on these two peaks leads to the thresholded computed-probability two-dimensional space shown in FIG. 17F. Note that the central feature pixels again can be readily distinguished from surrounding background pixels, despite the fact that, on the basis of pixel intensity, the feature pixels are not distinguishable from background pixels.

The above-employed examples are hypothetical examples, using computationally generated pixel intensities for hypothetical microarray-image regions. However, the variance-based pixel-partitioning technique described using these examples is equally effective when applied to actual microarray data. It has been observed that variance-based pixel partitioning can lead to more accurate feature-pixel/background-pixel partitioning than can be achieved by intensity-based partitioning methods. In commonly employed microarray data processing applications, feature pixels are distinguished from background pixels in several different channels, and a cumulative, multi-channel partitioning between feature pixels and the background pixels is employed in subsequent data processing. The cumulative, multi-channel partitioning may be based on the intersection of the feature masks generated for the microarray data for each channel.

It is desirable, from a computational standpoint, to employ the smallest possible number of bins in the computed-probability histogram in order to minimize the computational overhead for feature partitioning. However, noisy data often requires higher granularities of binning. Various different parameters may be computed to determine the proper initial binning level to be used in computing the first computed probability distribution histogram, in step 1406 of FIG. 14A. If, for example, the microarray-image region is known to contain a negative control, then a finer binning may be initially employed. If the signal-to-noise ratio for the microarray-image region, where the signal-to-noise region equals, is below a threshold value, than finer-granularity binning may be required. Finally, when an Euler number for the feature mask produced from the thresholded, two-dimensional, computed-probability space is less than zero, where the Euler number is the number of connected segments minus the number of holes in the mask, then finer binning may be justified.

Although the variance-based pixel partitioning has been described, in above examples, as being carried out over a microarray-image region containing a single feature, the method can be more generally applied to larger microarray-image regions. For example, the method may be applied to large microarray-image regions, or to the entire digital image of a microarray, in order to determine the boundaries of the feature-containing area of the microarray substrate so that the digital image can be suitably cropped for further data processing. In this application, a minimum-sized subimage containing greater than a threshold ration of feature pixels, possibly also constrained by geometric considerations, can be selected as the cropped digital image of the microarray substrate. Alternatively, horizontal and vertical projections of a two-dimensional probability space can be used to identify the boundary rows and columns of features, in a fashion analogous to using horizontal and vertical projections of a two-dimensional intensity space, as discussed in U.S. patent application Ser. No. 09/589,046, “Method and System for Extracting Data from Surface Array Deposited Features.” In another application, a larger region may be partitioned by the variance-based method that represents one embodiment of the present invention in order to provide an estimate of the ratio of feature pixels to background pixels, to enable calculation of an average feature size, in pixels, or radius in a convenient unit of length.

Although the present invention has been described in terms of a particular embodiment, it is not intended that the invention be limited to this embodiment. Modifications within the spirit of the invention will be apparent to those skilled in the art. For example, the variance-based method that represents one embodiment of the present invention may be applied to partition pixels into more than two sets, as described above. In the above examples, for instances, the variance of the central portion of the pixel differs from the variance of the annular, lower-intensity portion of the feature, which in turn differs from the variance of the background pixels. Thus, a two-threshold determination from three computed-probability distribution peaks could be employed to produce a thresholded image in computed probability space corresponding to the thresholded intensity maps in two-dimensional intensity space provided, for example, in FIGS. 15C and 16C. While the normal distribution assumption is found to be quite acceptable for computing the computed probability distribution histogram, any of many other, well known probability distributions and similar mathematical functions may also be used to transform two-dimensional intensity space into a two-dimensional computed-probability space. As greater experience is obtained in estimating the actual probability distributions of various digital images of microarrays, more accurate computed probability distributions may be obtained that provide more distinct peaks and a greater accuracy in distinguishing pixels having differential variances. And almost limitless number of different embodiments are possible, depending on in what medium the method is implemented and on details of implementation. For example, embodiments may be implemented in hardware, software, firmware, or a combination of two or more of hardware, software, and firmware, and software or logic may have many different modular organizations, use any of different control and data structures, and, in the case of software implementations, may be written in any of numerous different programming languages.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for partitioning pixels, each pixel associated with an intensity, in a subset of a microarray data set into a set of feature pixels and a set of background pixels, the method comprising:
computing parameters for a type of probability distribution to generate a particular probability distribution for the intensities associated with the pixels; and
partitioning the pixels by selecting as feature pixels those pixels associated with intensities having relatively low probabilities with respect to the generated probability distribution and selecting as background pixels those pixels associated with intensities having relatively high probabilities.

2. The method of claim 1 wherein the type of probability distribution is selected from among:
a Gaussian probability distribution;
a Poisson probability distribution;
a gamma probability distribution;
a Rayleigh probability distribution;
a chi-square probability distribution;
a beta probability distribution;
a binomial probability distribution; and
a probability distribution that models pixel-intensity distributions observed in sample scanned images of microarrays.

3. The method of claim 1 wherein computing parameters for the selected type of probability distribution to generate a particular probability distribution for the intensities associated with the pixels further comprises computing one or more parameters that define the particular probability distribution when the computed parameters are employed as constants within a mathematical expression by which probabilities of intensities are calculated.

4. The method of claim 3 wherein the computed parameters include a mean intensity and a standard deviation when the selected probability distribution is a normal distribution.

5. The method of claim 1 wherein partitioning the pixels by selecting as feature pixels those pixels associated with intensities having relatively low probabilities with respect to the generated probability distribution and selecting as background pixels those pixels associated with intensities having relatively high probabilities further includes:
for each pixel,
calculating a probability for the intensity associated with the pixel according to the generated probability distribution;
generating a computed probability space for the intensities of the pixels;
selecting a threshold that separates a first peak in the computed probability space from a second peak in the computed probability space; and
assigning pixels associated with intensities for which the calculated probabilities lie below the threshold in the computed probability space to the set of feature pixels, and assigning pixels associated with intensities for which the calculated probabilities lie above the threshold in the computed probability space to the set of background pixels.

6. The method of claim 5 wherein a probability for an intensity associated with a pixel is calculated, when the selected type of probability distribution is a normal distribution, by the expression:
$$p ( {I}_{i , j} ) = \frac{1}{\sqrt{2 π } σ} {ⅇ}^{- ( \frac{{( {I}_{i , j} - μ )}^{2}}{2 {σ}^{2}} )}$$
wherein
I_{i,j} is the intensity associated with the pixel at location (i,j) in the region of the scanned image of a microarray, the location (i,j) specified with respect to coordinate axes computed for the region;
σ is a standard deviation for the intensities associated with the pixels; and
μ is a mean for the intensities associated with the pixels.

7. The method of claim 5 wherein generating a computed probability space for the intensities of the pixels further includes generating a histogram for the computed probability space, each histogram bin containing a count of a number of pixels having computed intensity probabilities within a range of computed intensity probabilities associated with the histogram bin.

8. The method of claim 5 wherein selecting a threshold that separates a first peak in the computed probability space from a second peak in the computed probability space further includes:
identifying a first peak in the histogram and a second probability peak in the histogram; and
computing the threshold intensity probability so that pixel intensity probabilities of the first peak lie to a lower-probability side of the threshold intensity probability and pixels of the second peak lie to a higher-probability side of the threshold intensity probability.

9. The method of claim 8 wherein identifying a first peak in the histogram and a second probability peak in the histogram further includes applying a peak operator to local regions of each bin of the histogram to compute a peak metric for each bin and selecting two bins with highest peak metric values separated by at least a minimum threshold distance, in bins, from one another.

10. The method of claim 8 wherein applying a peak operator to a local region of a bin further includes summing differences between the count associated with the bin and the counts associated with neighboring bins in the local region.

11. The method of claim 8 wherein identifying a first peak in the histogram and a second probability peak in the histogram further includes selecting two bins with highest counts separated by at least a minimum threshold distance.

12. A method comprising forwarding to a remote location one of:
a pixel partitioning determined by the method of claim 1;
data obtained using a pixel partitioning determined by the method of claim 1; and
results obtained using a pixel partitioning determined by the method of claim 1.

13. A computer program implementing the method of claim 1 stored in a computer-readable medium.

14. A microarray data processing system that performs the method of claim 1.

15. A system for processing microarray data comprising pixels, each pixel associated with an intensity, the system comprising:
a processor; and
a program running on the processor that partitions the pixels into a set of feature pixels and a set of background pixels by
computing parameters for a type of probability distribution to generate a particular probability distribution for the intensities associated with the pixels; and
partitioning the pixels by selecting as feature pixels those pixels associated with intensities having relatively low probabilities with respect to the generated probability distribution and selecting as background pixels those pixels associated with intensities having relatively high probabilities.

16. The system of claim 15 wherein the type of probability distribution is selected from among:
a Gaussian probability distribution;
a Poisson probability distribution;
a gamma probability distribution;
a chi-square probability distribution;
a Rayleigh probability distribution;
a beta probability distribution;
a binomial probability distribution; and
a probability distribution that models pixel-intensity distributions observed in sample scanned images of microarrays.

17. The system of claim 15 wherein the program computes parameters for the type of probability distribution to generate a particular probability distribution for the intensities associated with the pixels by computing one or more parameters that define the particular probability distribution when the computed parameters are employed as constants within a mathematical expression by which probabilities of intensities are calculated.

18. The system of claim 17 wherein the computed parameters include a mean intensity and a standard deviation when the type of probability distribution is a normal distribution.

19. The system of claim 15 wherein the program selects as feature pixels those pixels associated with intensities having relatively low probabilities with respect to the generated probability distribution and selects as background pixels those pixels associated with intensities having relatively high probabilities by:
for each pixel,
calculating a probability for the intensity associated with the pixel according to the generated probability distribution;
generating a computed probability space for the intensities of the pixels;
selecting a threshold that separates a first peak in the computed probability space from a second peak in the computed probability space; and
assigning pixels associated with intensities for which the calculated probabilities lie below the threshold in the computed probability space to the set of feature pixels, and assigning pixels associated with intensities for which the calculated probabilities lie above the threshold in the computed probability space to the set of background pixels.

20. The system of claim 19 wherein the program calculates a probability for an intensity associated with a pixel, when the type of probability distribution is a normal distribution, by the expression:
$$p ( {I}_{i , j} ) = \frac{1}{\sqrt{2 π } σ} {ⅇ}^{- ( \frac{{( {I}_{i , j} - μ )}^{2}}{2 {σ}^{2}} )}$$
wherein
I_{i,j} is the intensity associated with the pixel at location (i,j) in the region of the scanned image of a microarray, the location (i,j) specified with respect to coordinate axes computed for the region;
σ is a standard deviation for the intensities associated with the pixels; and
μ is a mean for the intensities associated with the pixels.

21. The system of claim 19 wherein generating a computed probability space for the intensities of the pixels further includes generating a histogram for the computed probability space, each histogram bin containing a count of a number of pixels having computed intensity probabilities within a range of computed intensity probabilities associated with the histogram bin.

22. The system of claim 19 wherein selecting a threshold that separates a first peak in the computed probability space from a second peak in the computed probability space further includes:
identifying a first peak in the histogram and a second probability peak in the histogram; and
computing the threshold intensity probability so that pixel intensity probabilities of the first peak lie to a lower-probability side of the threshold intensity probability and pixels of the second peak lie to a higher-probability side of the threshold intensity probability.

23. A method for partitioning pixels, each pixel associated with an intensity, in a subset of a microarray data set into a set of feature pixels and a set of background pixels, the method comprising:
computing parameters for a type of probability distribution to generate a particular probability distribution for the intensities associated with the pixels;
generating a computed probability space by calculating the probabilities of the pixel intensities;
selecting thresholds intensity probabilities between peaks in the computed probability space, and
partitioning the pixels into sets having pixel-intensity probabilities between the selected thresholds.

24 A method comprising forwarding to a remote location one of:
a pixel partitioning determined by the method of claim 23;
data obtained using a pixel partitioning determined by the method of claim 23; and
results obtained using a pixel partitioning determined by the method of claim 23.

25. A computer program implementing the method of claim 23 stored in a computer-readable medium.

26. A microarray data processing system that performs the method of claim 23.

27. A method for partitioning pixels, each pixel associated with an intensity, in a subset of a microarray data set into a set of feature pixels and a set of background pixels, the method comprising:
for each data channel,
computing parameters for a type of probability distribution to generate a particular probability distribution for the intensities associated with the pixels, and
partitioning the pixels by selecting as feature pixels those pixels associated with intensities having relatively low probabilities with respect to the generated probability distribution and selecting as background pixels those pixels associated with intensities having relatively high probabilities; and
selecting as the set of feature pixels a combination of the selected feature pixels for each data channel.

28. The method of claim 27 wherein selecting as the set of feature pixels a combination of the selected feature pixels for each data channel further includes selecting as the set of feature pixels the set intersection of the feature pixels selected for each data channel.

29. The method of claim 27 wherein selecting as the set of feature pixels a combination of the selected feature pixels for each data channel further includes:
for each data channel,
computing a mask based on a probability distribution discriminator;
generating a cumulative mask from the masks computed for each data channel; and
selecting feature and background pixels by using the generated cumulative mask.

30 A method comprising forwarding to a remote location one of:
a pixel partitioning determined by the method of claim 23;
data obtained using a pixel partitioning determined by the method of claim 23; and
results obtained using a pixel partitioning determined by the method of claim 23.

31. A computer program implementing the method of claim 23 stored in a computer-readable medium.

32. A microarray data processing system that performs the method of claim 23.

33. A method for cropping a digital image of a microarray to produce a subimage containing feature pixels and a background subimage surrounding the subimage containing features, the method comprising:
computing parameters for a type of probability distribution to generate a particular probability distribution for the intensities associated with the pixels, and
partitioning the pixels by selecting as feature pixels those pixels associated with intensities having relatively low probabilities with respect to the generated probability distribution and selecting as background pixels those pixels associated with intensities having relatively high probabilities; and
cropping the digital image using the pixel partitioning.

34. The method of claim 33 wherein cropping the digital image using the pixel partitioning further includes:
selecting as the subimage containing feature pixels the smallest subimage containing greater than a threshold percentage of the feature pixels.

35. The method of claim 33 wherein cropping the digital image using the pixel partitioning further includes:
using horizontal and vertical projections of a two-dimensional probability space obtained by computing the probabilities of pixel intensities according to the generated probability distribution to locate the boundary feature rows and feature columns of the digital image and cropping the digital image to include the boundary feature rows and feature columns of the digital image and interior rows and columns.

36 A method comprising forwarding to a remote location one of:
a subimage containing feature pixels determined by the method of claim 33;
data obtained using a subimage containing feature pixels determined by the method of claim 33; and
results obtained using a a subimage containing feature pixels determined by the method of claim 33.

37. A computer program implementing the method of claim 33 stored in a computer-readable medium.

38. A microarray data processing system that performs the method of claim 33.
